(19) United States
(12) Patent Application Publication
(10) Pub. No.: US 2008/0097306 A1
(43) Pub. Date: Apr. 24, 2008

(54) Sterilized syringe

(76) Inventors: Jeffrey Smith, Irvine, CA (US); Daniel Thayer, Mission Viejo, CA (US); Rex O. Bare, Irvine, CA (US); Steve Kampff, Newport Beach, CA (US)

Correspondence Address: STETINA BRUNDA GARRED & BRUCKER, 75 ENTERPRISE, SUITE 250, ALISO VIEJO, CA, 92656, US

(21) Appl. No.: 11/511,668

(22) Filed: Aug. 29, 2006

(51) Int. Cl. A61M 5/00; A61B 19/00

(52) U.S. Cl. 604/110; 604/171; 128/898

(57) ABSTRACT

A method of maintaining sterility of fluidic medication in a fluidic medication container despite repeated extractions of the fluidic medication is provided. Also, a method of injecting a patient with fluidic medication without exposing the fluidic medication to ambient air is provided. The method may comprise the steps of providing a syringe with a sterilized variable fluid chamber with sterilized gas wherein the syringe is in a retracted position. A needle of the syringe is inserted into the fluidic medication container and the sterilized gas in the sterilized variable fluid chamber is transferred into the fluidic medication container to pressurize the same. Thereafter, fluidic medication is transferred from the fluidic medication container into the sterilized variable fluid chamber. The needle is removed from the fluidic medication container and inserted into the patient. The patient is injected with the fluidic medication in the sterilized variable fluid chamber.

### CROSS-REFERENCE TO RELATED APPLICATIONS

This application is related to U.S. Pat. No. 6,413,236, filed Jun. 20, 2000, the entire content of which is expressly incorporated herein by reference.

### STATEMENT RE: FEDERALLY SPONSORED RESEARCH/DEVELOPMENT

Not Applicable

### BACKGROUND

The present invention relates to maintaining a sterile environment in which fluidic medication travels from a medication vial to a syringe and ultimately to the patient. Also, the present invention relates to maintaining sterility of fluidic medication contained in a sterilized medication vial such that the sterilized medication vial may be repeatedly extracted and none of the fluidic medication wasted.

Prior art syringes are sterilized before they are provided to medical professionals or users to prevent infections caused by contaminants that may reside in the prior art syringe and be ultimately injected into the patient along with the fluidic medication. Unfortunately, the process of injecting the patient with fluidic medication via prior art sterilized syringes still creates an inherent risk of contaminating the fluidic medication and injecting the patent with contaminated fluidic medication. In particular, during the process of injecting fluidic medication into the patient, the risk of contaminating the fluidic medication to be injected occurs when the fluidic medication is exposed to ambient air or surfaces exposed to ambient air. The fluidic medication “picks up” contaminants when the fluidic medication is exposed to ambient air or surfaces exposed to ambient air because viruses, bacteria and other unwanted contaminants may be in the ambient air.

For example, all prior art syringes are provided to medical professionals or users with a piston closely adjacent to a distal end of the syringe body. At this point, the syringe is sterile. During operation of the prior art syringe, the piston must be initially traversed away from the distal end of the syringe body and toward the proximal end of the syringe body. Such traversal of the piston away from the distal end of the syringe body increases a volume of a variable fluid chamber and simultaneously introduces ambient air into the variable fluid chamber.

Unfortunately, the ambient air introduced into the variable fluid chamber may contain viruses, bacteria or other unwanted contaminants. The viruses, bacteria and unwanted contaminants may be lodged onto the surfaces of the variable fluid chamber. Now, the variable fluid chamber of the prior art syringe is unsterilized or contaminated. Fluidic medication extracted from a medication vial is held in the contaminated variable fluid chamber just prior to injection into the patient, and thus, the fluidic medication to be injected into the patient is contaminated.

The fluidic medication to be injected into the patient may also be contaminated before the fluidic medication is introduced into the variable fluid chamber. In particular, as stated above, the piston of the prior art syringe is traversed away from the distal end of the syringe body. This introduces ambient air, which may contain contaminants, into the variable fluid chamber. The contaminated ambient air in the variable fluid chamber is then transferred into the medication vial by inserting the syringe needle into the medication vial and traversing the piston toward the distal end of the syringe body to pressurize the medication vial. Beneficially, the increased pressure assists in withdrawal of the fluidic medication from the medication vial. Unfortunately, the contaminated ambient air is now inside the medication vial, and thus, the fluidic medication in the medication vial has become contaminated.

The fluidic medication in the medication vial which has become contaminated is now transferred into the variable fluid chamber. In particular, the medication vial and the prior art syringe are inverted. The tip of the syringe needle is positioned within the contaminated fluidic medication. The piston of the syringe is traversed away from the distal end of the syringe body. The increased pressure in the medication vial and the traversal of the piston transfers the contaminated fluidic medication into the variable fluid chamber.

Accordingly, the fluidic medication to be injected into the patient may become contaminated at two different stages. First, the fluidic medication may become contaminated when contaminated air is introduced into the medication vial. Second, the fluidic medication may become contaminated when the fluidic medication is transferred into the contaminated variable fluid chamber.

In view of the above discussion, the current practice of hospitals, doctors, nurses is to discard any remaining fluidic medication in the medication vial after a single does of fluidic medication has been withdrawn from the medication vial. The reason is that repeatedly introducing contaminated ambient air into the medication vial to pressurize the medication vial increases the number and types of viruses, bacteria and other unwanted contaminants into the fluidic medication. Unfortunately, the current practice wastes potentially expensive fluidic medication. For example, if the medication vial contained a sufficient amount of fluidic medication for four doses but the patient only required one dose, then the remaining three doses would be discarded due to contamination of the remaining fluidic medication when the first dose was withdrawn. This practice wastes the remaining three doses of fluidic medication. In expensive treatments, this practice may cost the hospital a substantial amount of money.

In sum, the current practice of injecting fluidic medication wastes fluidic medication. Also, in prior art syringes, the basic operation of the syringe creates an inherent risk of injecting the patient with contaminated fluidic medication.

### BRIEF SUMMARY

The syringe and the method discussed herein addresses the deficiencies in the art discussed above, below and those that are known in the art.

The method may comprise the steps of providing a syringe with a sterilized variable fluid chamber with sterilized air contained therein. The syringe may be disposed within a hermetically sealed package with the syringe at a retracted position or the piston positioned away from the distal end of the syringe body. Once the syringe is transported to a user or medical professional, the user or medical professional may remove the syringe from the package. At this point, the ambient air is not mixed with the sterilized air in the sterilized variable fluid chamber or introduced into the sterilized variable fluid chamber. The user inserts a needle of the syringe into a fluidic medication container and transfers the sterilized air in the sterilized variable fluid chamber into the fluidic medication container thereby pressurizing the fluidic medication container. The piston did not have to be traversed rearwardly away from the distal end of the syringe body to introduce contaminated ambient air into the variable fluid chamber. Sterilized air to be injected into the medication vial was already in the variable fluid chamber. A distal tip of a needle of the syringe is then positioned within the fluidic medication of the fluidic medication container. The syringe is then traversed back toward the retracted position. Traversal of the syringe back toward the retracted position and the pressure within the fluidic medication container urges the fluidic medication in the fluidic medication container through the needle and into the sterilized variable fluid chamber. The needle of the syringe is removed from the fluidic medication container and is inserted into the body of the patient. The fluidic medication is then injected into the patient. Thereafter, the needle is removed from the patient and the syringe is discarded.

Accordingly, the fluidic medication extracted from the fluidic medication container has not been exposed to ambient air or surface exposed to ambient air throughout the entire process of injecting the patient with the fluidic medication, and thus, the fluidic medication injected into the patient is sterile. Also, the fluidic medication container may be repeatedly accessed to extract fluidic medication to deplete all of the fluidic medication in the fluidic medication container.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a top view of a syringe disposed in a package;

FIG. 2 is a flow chart of a method of maintaining sterility of fluidic medication in a fluidic medication container and injecting a patient with fluidic medication in a sterile manner;

FIG. 3 is an illustration of a fluidic medication container containing fluidic medication and a syringe in a retracted position wherein a needle of the syringe is to be inserted into the fluidic medication container;

FIG. 4 is an illustration of the syringe needle inserted into the fluidic medication container and sterilized air in the sterilized variable fluid chamber transferred into the fluidic medication container to pressurize the same;

FIG. 5 is an illustration of fluidic medication being withdrawn into the sterilized variable fluid chamber; and

FIG. 6 is an illustration of a patient being injected with the fluidic medication.

### DETAILED DESCRIPTION

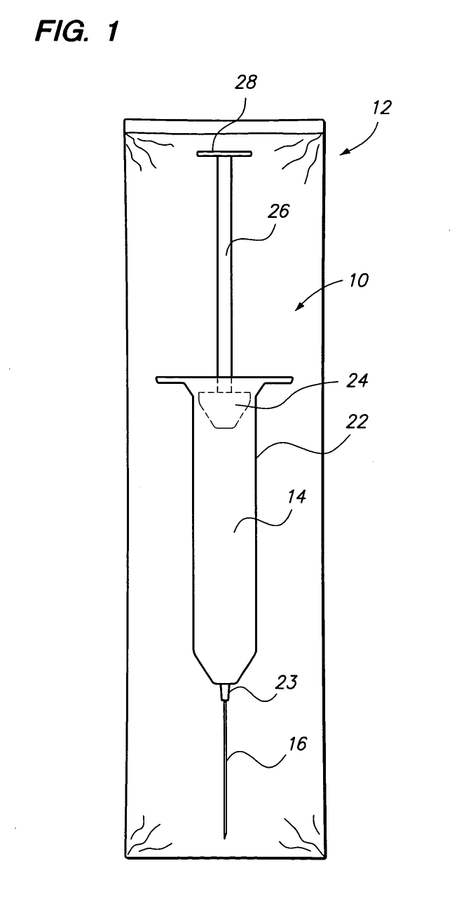

Referring now to the drawings which are for the purpose of illustration and not for the purpose of limiting the same, FIG. 1 illustrates a syringe 10 disposed in a hermetically sealed package 12. The syringe 10 may be any one of a plurality of different types of syringes such as a standard syringe or an automatically retractable safety syringe (discussed later). The contents of the hermetically sealed package 12 including the syringe 10 may be sterilized. In particular, the gas (e.g., air) contained within the hermetically sealed package 12 and within a variable fluid chamber 14 of the syringe 10 may be sterilized. Moreover, the surfaces, preferably, the internal surface of the hermetically sealed package 12 as well as the needle 16 and the surfaces defining the variable fluid chamber 14 may be sterilized. Preferably, all surfaces of the syringe 10 may be sterilized prior to packaging the syringe 10 in the hermetically sealed package 12. This sterilized syringe 10 may be provided to the medical professional or user for injecting fluidic medication 18 into a patient 20.

The syringe 10 disposed in the hermetically sealed package 12 may be sterilized as follows. First, a plurality of syringe components (e.g., syringe body 22, needle 16, needle holder 23, piston 24, plunger shaft 26, thumb platform 28 and seals (not shown)) may be provided. These components of the syringe 10 may be fabricated via any process known in the art such as injection molding and with any material suitable for use as syringe components. After the syringe components are fabricated, the syringe components may be sterilized in a clean room environment as well as assembled in the clean room environment. At this point, the variable fluid chamber 14 of the syringe 10 is sterile in that the surfaces defining the variable fluid chamber 14 as well as the gas (e.g., air) in the variable fluid chamber 14 is sterile. A plurality of plastic packages 12 may be provided and one sterilized syringe 10 may be inserted into each package 12. These packages 12 may also be sterilized such that the sterilized syringe 10 may not become subsequently contaminated. The packages 12 are then hermetically sealed to ensure that the sterilized syringe 10 remains sterile when the sterilized syringe 10 is transported from the manufacturer to the medical professional or user.

As shown in FIG. 1, the syringe 10 may be provided to the medical professional with the piston 24 in the retracted position. As such, the air within the variable fluid chamber 14 is sterile and the surfaces of the syringe 10 defining the variable fluid chamber 14 is also sterile when the syringe 10 is removed from the package 12. When the needle 16 is inserted into the fluidic medication container 30 and the sterile air within the variable fluid chamber 14 is transferred into the fluidic medication container 30 to pressurize the same and to assist in withdrawal of the fluidic medication 18 from the fluidic medication container 30 to the variable fluid chamber 14, the fluidic medication container 30, and more particularly, the contents within the fluidic medication container 30 remains sterile because only sterilized air (i.e., sterilized air contained within the variable fluid chamber 14) is used to pressurize the fluidic medication container 30, as shown in FIG. 4. In this manner, the inner contents of the fluidic medication container 30, namely, the gas (e.g., air) within the fluidic medication container 30 as well as the fluidic medication 18 itself is not contaminated. As such, the fluidic medication 18 remaining in the fluidic medication container 30 may be repeatedly extracted via the steps discussed herein until all of the fluidic medication 18 has been dispensed.

In a first aspect, the fluidic medication 18 injected into the patient 20 does not come into contact with ambient air throughout the entire process of injecting fluidic medication 18 into the patient 20. As discussed above, when the syringe 10 is packed in the hermetically sealed package 12, the syringe 10 and all surfaces are sterilized. Additionally, the syringe 10 may be packed in the hermetically sealed package 12 while in a clean room environment. As such, the gas within the hermetically sealed package 12 and within the variable fluid chamber 14 may be characterized as being sterilized. Additionally, the components of the syringes 10 themselves may also be characterized as being sterile.

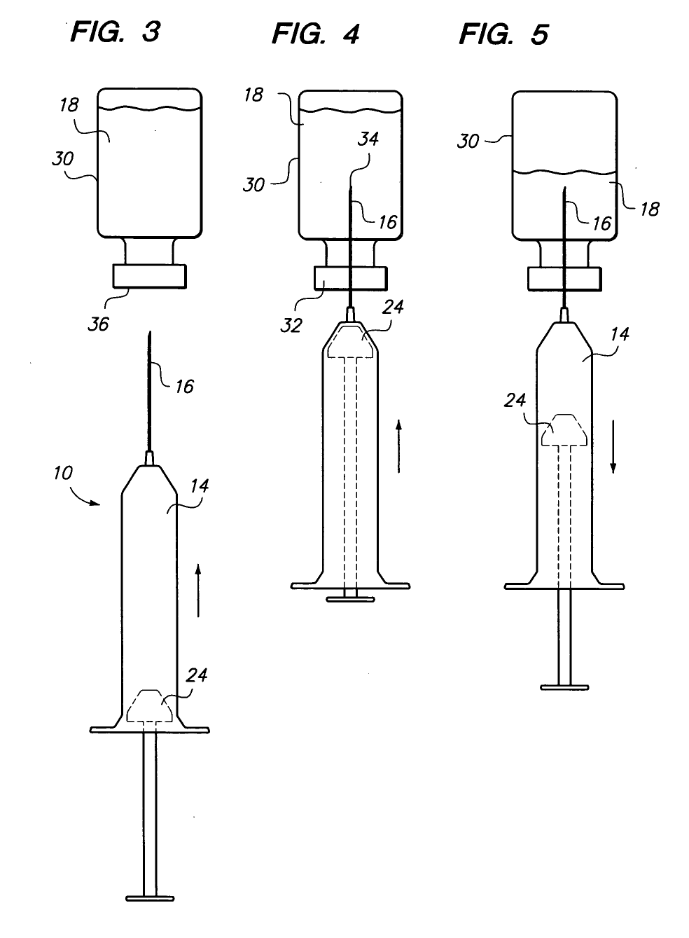

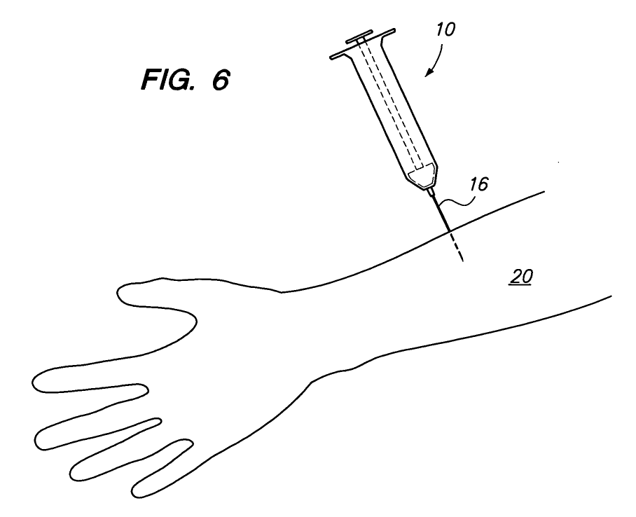

To inject the patient 20 (see FIG. 6) with fluidic medication 18, the syringe 10 is initially removed from the hermetically sealed package 12. Once the syringe 10 is removed from the hermetically sealed package 12, the exterior surfaces of the syringe 10 are exposed to ambient air. Fortunately, the sterile gas or air within the variable fluid chamber 14 is not mixed with the ambient air. Ambient air is not introduced into the variable fluid chamber 14. Simply put, the variable fluid chamber 14 and the gas contained therein remains sterile. After the syringe 10 has been removed from the hermetically sealed package 12, the needle 16 is inserted into the fluidic medication container 30. More particularly, the needle 16 is inserted through a rubber stopper 32 of the fluidic medication container 30 and a distal tip 34 of the needle 16 is positioned within the fluidic medication 18 contained in the fluidic medication container 30, as shown in FIG. 4. Optionally, the exterior surface 36 (see FIG. 3) of the rubber stopper 32 may be sterilized before the needle 16 is pierced therethrough. For example, a disinfecting cloth may be used to wipe the exterior surface 36 of the rubber stopper 32. After the needle distal tip 34 is positioned in the fluidic medication container 30, the medical professional or user traverses the piston 24 toward the filling position (see FIG. 4) which transfers the sterile air in the variable fluid chamber 14 into the fluidic medication container 30 and pressurizes the same, as shown in FIG. 4. With the fluidic medication container 30 pressurized and the distal tip 34 of the needle 16 positioned within the fluidic medication 18 contained in the fluidic medication container 30, the medical professional or user traverses the piston 24 toward the retracted position, as shown in FIG. 5. The traversal of the piston 24 toward the retracted position transfers fluidic medication 18 from the fluidic medication container 30 into the variable fluid chamber 14 and the pressure in the fluidic medication container 30 assists in such transfer. Thereafter, the needle 16 is removed from the fluidic medication container 30 and inserted into the body of the patient 20, as shown in FIG. 6. The fluidic medication 18 is injected into the patient 20 by traversing the piston 24 from the retracted position toward the dispensed position (see FIG. 6). Once the fluidic medication 18 is administered or injected into the patient 20, the needle 16 is removed from the patient 20 and discarded in a safe manner.

Accordingly, the fluidic medication 18 injected into the patient does not come into contact with ambient air during operation of the syringe 10. Also, the inner contents of the fluidic medication container 30 was not exposed to ambient air during extraction of the fluidic medication 18. Accordingly, the remaining fluidic medication 18 in the fluidic medication container 30 may be subsequently extracted for administration or injection into a patient 20 until all of the fluidic medication 18 is dispensed. For expensive medication, dispensing all the fluidic medication 18 in the fluidic medication container 30 may be a substantial savings to the hospital, medical professional, user or purchaser of the fluidic medication 18.

In sum, there are two beneficial aspects of the above described process, first, the fluidic medication 18 in the fluidic medication container 30 may be fully dispensed thereby saving the purchaser of the fluidic medication 18 a substantial amount of money. Also, the fluidic medication 18 injected into the patient 20 is not exposed to ambient air during the entire process of extracting the fluidic medication 18 into the fluidic medication container 30 and ultimately injecting the fluidic medication into the patient 20.

In another aspect, the syringe 10 may be a specialty syringe. For example, the syringe 10 may be an automatically retractable safety syringe. One type of automatically retractable safety syringe is described in U.S. Pat. No. 6,413,236, issued to Van Dyke, the entire contents of which is expressly incorporated herein by reference. The retractable safety syringe 10 may comprise a needle 16, needle holder 23, syringe body 22, piston 24, plunger shaft 26 and thumb platform 28 as well as a piston seal (not shown) and a shaft seal (not shown). The retractable safety syringe 10 may have a variable fluid chamber 14 defined by the syringe body 22, needle holder 23 and piston 24. Also, on the opposed side of the piston 24, the retractable safety syringe 10 may have a variable vacuum compartment. The variable vacuum compartment may be defined by the piston seal, shaft seal and the syringe body 22. The variable vacuum compartment assists in retracting or urging the piston 24 back toward a retracted position during operation of the retractable safety syringe 10.

The retractable safety syringe 10 may be sterilized and packaged in a hermetically sealed package 12. Initially, the piston 24 of the automatic retractable syringe 10 may be at the retracted position (see FIG. 1). The syringe 10 itself as well as the air or gas in the variable fluid chamber 14 and the hermetically sealed package 12 may also be sterile by packaging sterilized components of the retractable safety syringe 10 in a clean room environment, as discussed above.

To inject fluidic medication 18 into a patient 20, the retractable safety syringe 10 may be removed from the hermetically sealed package 12, as shown in FIG. 3. At this point, the exterior surfaces of the retractable safety syringe 10 are exposed to ambient air. Importantly, the variable fluid chamber 14 and the gas or air contained therein is not mixed with ambient air and ambient air is not introduced into the variable fluid chamber 14. The needle 16 of the retractable safety syringe 10 is inserted into the fluidic medication container 30 through the rubber stopper 32 of the fluidic medication container 30. Optionally, the exterior surface 36 (see FIG. 3) of the rubber stopper 32 may be disinfected prior to insertion of the needle 16 therethrough. For example, the exterior surface 36 of the rubber stopper 32 may be disinfected with a disinfecting cloth. After the needle 16 is inserted into the fluidic medication container 30, the sterilized air in the variable fluid chamber 14 is transferred into the fluidic medication container 30 to pressurize the same 30, as shown in FIG. 4. The distal tip 34 of the needle 16 of the retractable safety syringe 10 is positioned within the fluidic medication 18 of the fluidic medication container 30 and the piston 24 is traversed back toward the retracted position, as shown in FIG. 5. The pressure in the fluidic medication container 30 and traversal of the piston 24 toward the retracted position transfers fluidic medication 18 from the fluidic medication container 30 into the variable fluid chamber 14. At no time was the fluidic medication 18 exposed to ambient air. The needle 16 may now be removed and inserted into the patient 20. The fluidic medication 18 is now injected into the patient 20, as shown in FIG. 6. The needle 16 is removed from the patient 20 and the syringe 10 discarded. At no point during the process of injecting the fluidic medication 18 into the patient 20 was the injected fluidic medication 18 exposed to ambient air. As such, the above mentioned process ensures that the injected fluidic medication 18 is not contaminated.

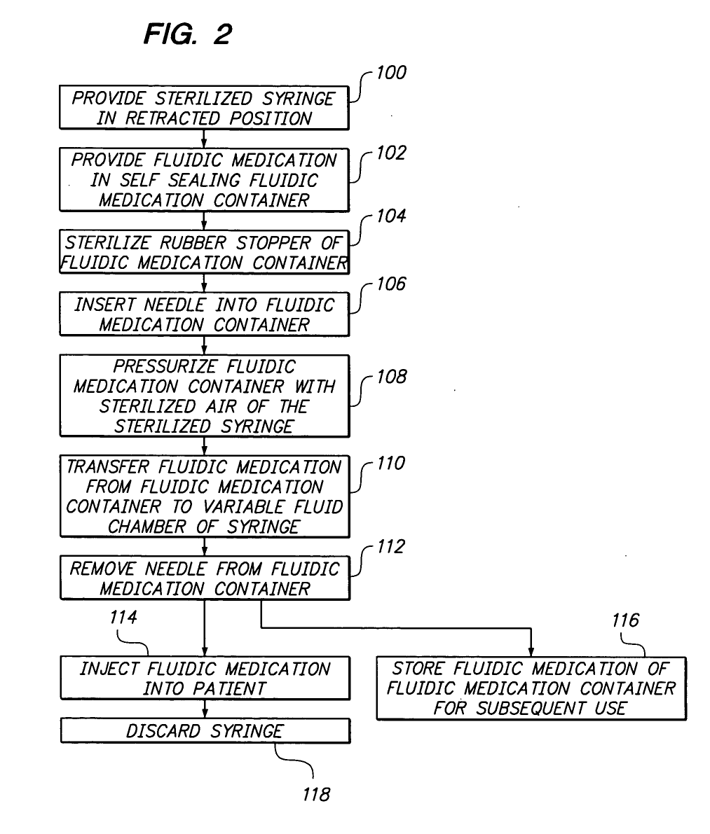

In an aspect, as shown in FIG. 2, a method is provided wherein fluidic medication 18 contained within a sterilized medication container is withdrawn from the fluidic medication container 30 and injected into a patient 20 without the injected fluidic medication 18 being exposed to ambient air. As such, the fluidic medication 18 remains sterile throughout the entire process of injecting the patient 20 with the fluidic medication 18. Additionally, in another aspect of the method, the fluidic medication 18 within the fluidic medication container 30 is not exposed to ambient air when the fluidic medication 18 is extracted from the fluidic medication container 30 via a syringe 10. In this manner, the fluidic medication 18 in the fluidic medication container 30 remains sterile even though the fluidic medication 18 in the fluidic medication container 30 may be repeatedly accessed or extracted each time a patient 20 is to be injected with the fluidic medication 18 such that all of the fluidic medication 18 in the fluidic medication container 30 is depleted.

In the method, a first step may be providing 100 a sterilized syringe 10 in a retracted position. The syringe 10 may be sterilized by sterilizing each of the components of the syringe 10 and assembling the syringe components in a clean room environment such that a variable fluid chamber 14 and gas in the variable fluid chamber 14 is sterile. In a second step, fluidic medication 18 may be provided 102 in a self-sealing fluidic medication container 30. Before the needle 16 of the syringe 10 is inserted into the fluidic medication container 30, a rubber stopper 32 of the fluidic medication container 30 may be sterilized 104. For example, a disinfecting cloth may wipe the exterior surface 36 of the rubber stopper 32 to sterilize the rubber stopper 32. After the rubber stopper 32 is sterilized, the needle 16 of the syringe 10 may be inserted 106 into the fluidic medication container 30 through the rubber stopper 32. The fluidic medication container 30 may be pressurized 108 with the sterilized air of the sterilized variable fluid chamber 14. In particular, after the needle 16 is inserted into the fluidic medication container 30, the syringe 10 is traversed toward the filling position. In this manner, the sterilized air in the variable fluid chamber 14 is transferred from the variable fluid chamber 14 into the fluidic medication container 30. After the fluidic medication container 30 is pressurized, the fluidic medication 18 may be transferred 110 from the fluidic medication container 30 to the variable fluid chamber 14 of the syringe 10. In particular, a distal tip 34 of the needle 16 is positioned within the fluidic medication 18 in the fluidic medication container 30, as shown in FIG. 4. The syringe 10 is then traversed back toward the retracted position. The pressure within the fluidic medication container 30 and traversal of the syringe 10 back toward the retracted position urges the fluidic medication 18 through the needle 16 and into the variable fluid chamber 14. After the appropriate amount of fluidic medication 18 is transferred into the variable fluid chamber 14, the needle 16 is removed 112 from the fluidic medication container 30. At this point, the fluidic medication 18 is injected 114 into the patient 20 by inserting the needle 16 into the patient 20 and traversing the syringe 10 toward the engaged or dispensing position. The needle 16 is then removed from the patient 20 and the syringe 10 discarded 118. Also, the fluidic medication 18 of the fluidic medication container 30 is stored 116 for subsequent use.

As used herein, the retracted position of the syringe 10 is where the piston 24 is distanced from the needle 16. Typically, the piston 24 is closely adjacent to the proximal end of the syringe body 22. The filling position of the syringe 10 refers to the syringe 10 before fluidic medication 18 has been filled into the syringe 10. The piston 24 is closer to the distal end of the syringe body 22 than to the proximal end of the syringe body 22. More particularly, the piston 24 is closely adjacent to the distal end of the syringe body 22. The dispensed position of the syringe 10 refers to the syringe 10 after fluidic medication 18 has been filled into the variable fluid chamber 14 and the fluidic medication 18 has been injected into the patient 20. The piston 24 is closely adjacent to the distal end of the syringe body 22 when the syringe 10 is at the dispensed position.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of injecting a patient in a sterile manner, the method comprising the steps of:
a. providing a syringe in a retracted position with sterilized gas within a sterilized variable fluid chamber;
b. inserting a needle of the syringe in a sterile fluidic medication container;
c. traversing the syringe toward a filling position thereby transferring the sterilized gas of the variable fluid chamber into the sterile fluidic medication container and pressuring the sterile fluidic medication container;
d. traversing the syringe toward the retracted position thereby transferring fluidic medication contained within the sterile fluidic medication container into the sterilized variable fluid chamber;
e. removing the needle of the syringe from the sterile fluidic medication container; and
f. injecting the patient with the fluidic medication transferred into the sterilized variable fluid chamber.

2. The method of claim 1 wherein the inserting step is characterized by inserting the needle of the syringe through a self sealing rubber stopper of the sterile fluidic medication container.

3. The method of claim 1 further comprising the steps of:
a. disposing the retractable safety syringe in a package with the syringe in the retracted position; and
b. sterilizing an inner volume of the package.

4. The method of claim 1 wherein in the providing step, the retractable safety syringe is normally in the retracted position.

5. The method of claim 1 wherein the providing step further includes the step of providing a retractable safety syringe.

6. A method of maintaining sterility of a fluidic medication container despite repeated extractions of fluidic medication from the fluidic medication container, the method comprising the steps of:
a. providing a plurality of sterilized syringes in a retracted position with sterilized gas in a sterilized variable fluid chamber;
b. providing a sterile fluidic medication container wherein the container comprises a vial, fluidic medication contained within the vial, and a self sealing rubber stopper to contain the fluidic medication within the vial;
c. extracting fluidic medication from the vial via a first sterilized syringe, the extracting step comprising the steps of:
inserting a needle of the first sterilized syringe into the sterile fluidic medication container;
transferring the sterilized gas of the sterilized variable fluid chamber of the first sterilized syringe into the sterile fluidic medication container;
transferring the fluidic medication of the sterile fluidic medication container into the sterilized variable fluid chamber of the first sterilized syringe;
removing the needle of the first sterilized syringe from the sterile fluidic medication container;
d. extracting fluidic medication from the via a second sterilized syringe, the extracting step comprising the steps of:
inserting a needle of the second sterilized syringe into the sterile fluidic medication container;
transferring the sterilized gas of the sterilized variable fluid chamber of the second sterilized syringe into the sterile fluidic medication container;
transferring the fluidic medication of the sterile fluidic medication container into the sterilized variable fluid chamber of the second sterilized syringe;
removing the needle of the second sterilized syringe from the sterile fluidic medication container.

7. A device for maintaining sterility of a fluidic medication container and for injecting a patient with fluidic medication without exposing the fluidic medication to ambient air, the syringe comprising:
a syringe having a sterilized variable fluid chamber with sterile air contained in the sterilized variable fluid chamber; and
a hermetically sealed package sized and configured to receive the syringe, the package having a sterilized inner surface and sterile air contained in the package;
wherein the syringe is disposed within the hermetically sealed package with the syringe at a retracted position for providing sterilized air within the sterilized variable fluid chamber to be transferred into the sterile fluidic medication container.

8. The device of claim 7 wherein the syringe is a vacuum operated automatic retractable safety syringe.
